(19) United States
(12) Patent Application Publication
(10) Pub. No.: US 2005/0107474 A1
(43) Pub. Date: May 19, 2005

(54) Pharmaceutical compositions for the treatment of infections of the respiratory system by pathogenic agents

(76) Inventors: Domenico Ungheri, Parabiago (IT); Luciano Licciardello, Monza (IT); Giovanni Battista Colombo, Cassina De Pecchi (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/486,929

(22) PCT Filed: Jun. 19, 2003
(86) PCT No.: PCT/EP03/06454

(30) Foreign Application Priority Data
Jun. 25, 2002 (IT) MI 2002A 001405

(51) Int. Cl. A61K 31/198; A61K 31/16

(52) U.S. Cl. 514/562; 514/599

(57) ABSTRACT

The present invention relates to pharmaceutical compositions for the treatment of infections by pathogen agents of the respiratory system, for example Staphylococcus aureus, and more particularly it relates to the aforesaid compositions containing N-acetyl-cysteine possibly combined with thiamphenicol.

The present invention relates to pharmaceutical compositions for the treatment of infections by pathogen agents of the respiratory system and, more particularly, it relates to the aforesaid compositions containing N-acetyl-cysteine possibly combined with thiamphenicol.

N-acetyl-cysteine (hereinafter NAC) is a known drug with mucolytic and antioxidant activity which is sold for example in Italy with the trade mark FLUIMUCIL of Zambon Italia S.r.l.

Thiamphenicol (hereinafter TAPh) is a known antibiotic which is sold in Italy as glycinate hydrochloride with the trade mark GLITISOL of Zambon Italia S.r.l.

The same Company is currently marketing the drug FLUIMUCIL ANTIBIOTIC that contains both of the aforementioned active principles, being constituted of thiamphenicol glycinate acetylcysteinate.

Various pathogen agents of the respiratory system, for example Staphylococcus aureus, are able to produce an abundant extracellular mucopolysaccharide substance which promotes bacterial adhesiveness and the production of biofilms.

Biofilms often enable bacteria to colonize external surfaces such as prostheses or catheters.

In a biofilm, the bacteria can develop in an environment that is protected both against immune responses and against antibiotics.

Biofilms thus represent an important problem in antibiotic therapy and are often the cause of recurrent infections.

Clearly, therefore, there is a need for substances that are able to inhibit the formation of biofilms or disrupt existing biofilms.

NAC has been recognized as having the ability to disrupt biofilms of P. aeruginosa (Antimicrob. Agents Chemother., 35, 1258, (1991)) and to inhibit their formation by S. epidermidis (J. Ant. Chem., 39, 643, (1997)).

We have now found that NAC is able both to inhibit the formation of biofilms by pathogen agents of the respiratory system and in particular by S. aureus, and to disrupt them if already formed.

From now on our discussion will focus on S. aureus, it being understood that what is stated also applies to other pathogen agents of the respiratory system that are able to produce biofilms.

In addition, we have found that TAPh is able to enhance the activity of NAC in inhibiting the formation of biofilms of S. aureus or disrupting them.

Therefore a first object of the present invention is a process for preparing a pharmaceutical composition containing N-acetyl-cysteine for the preventive or disruptive treatment of biofilms produced by Staphylococcus aureus or by other pathogen agents of the respiratory system.

A second object of the present invention is a process for preparing a pharmaceutical composition containing thiamphenicol useful for increasing the activity of N-acetyl-cysteine in inhibiting or disrupting the biofilm produced by Staphylococcus aureus and by other pathogen agents of the respiratory system.

A third object of the invention is a pharmaceutical composition containing N-acetyl-cysteine and thiamphenicol and use thereof in inhibiting or disrupting biofilms produced by Staphylococcus aureus and by other pathogen agents of the respiratory system.

Finally, a fourth object of the invention is a process for preparing a pharmaceutical composition containing thiamphenicol glycinate acetylcysteinate useful for inhibiting or disrupting biofilms produced by Staphylococcus aureus and by other pathogen agents of the respiratory system.

The aforesaid compositions permit successful attack of Staphylococcus aureus even when it is protected by biofilm.

The compositions of the invention are prepared in accordance with traditional methods using additives that are already known and used in the pharmaceutical sector.

Depending on the chosen route of administration, they can be in the form of aerosol vials, injectable vials and solid compositions for oral use such as capsules, tablets and effervescent tablets.

If we wish to treat infections of S. aureus in the lungs the preferred route of administration will be aerosol and therefore the preferred compositions are solutions that can be administered in the airways.

The compositions can be used both for a preventive treatment when, suspecting or having demonstrated S. aureus infection, we wish to prevent this creating the biofilm in which it will then be difficult to attack it with a conventional antibiotic treatment.

The compositions can also be used as co-adjuvants in a conventional antibiotic treatment when this proves insufficiently effective on account of the protective action of the biofilm on the microorganism.

The effective doses of NAC are between 200 and 1800 mg/day to be taken in one or more doses. Treatment of the airways can use aerosol solutions at a concentration of 10%.

TAPh will generally be administered by injection or as aerosol; in the latter case, 2 ml of 5% solution will be used one or more times a day.

Thiamphenicol glycinate acetylcysteinate will also preferably be administered as injectable solution or with aerosol using concentrations equivalent to 500 mg of TAPh.

Of course, the doses stated above are those preferred because they refer to products already being marketed, although medical practice can vary the dosages and times, without departing from the spirit of the invention.

With the aim of better illustrating the present invention though without limiting it, the following examples are now provided.

The effects of NAC and TAPh on the biofilm produced by S. aureus were evaluated on four strains of the microorganism designated 1393, 1876, 1880 and 1890.

### EXAMPLE 1

### Formation and Quantification of the Biofilm

Formation of the biofilms was quantified spectrophotometrically in polystyrene microplates with U-well geometry (Corning Incorporated, New York).

In more detail, the effect of NAC on biofilm formation was evaluated as follows: overnight broth cultures in tryptic soy broth (TSB) with glucose added (0.25%) (TSBG) were diluted 1:100 in TSBG. Appropriate dilutions of NAC at concentrations varying respectively from 8 mg/ml to 0.007 mg/ml were added to the suspensions (100 μl each). After incubation at 37° C. for 24 hours, the microplates were washed three times with 10 mM phosphate buffer pH 7.4 (PBS), treated with Bouin fixative and then stained with crystal violet (0.01%).

The biofilm adhering to the walls of the microplates was washed with PBS, suspended in 10% sarcosyl and measured spectrophotometrically at 492 nm.

The same technique was employed for calculating the residual quantity of biofilm after treatment with NAC alone and combined with TAPh on S. aureus. For this purpose, biofilms with two different degrees of maturity: initial (5 hours) and fully consolidated (48 hours) were exposed to suitable concentrations of NAC (from 8 mg/ml to 0.007 mg/ml) with added TAPh at the concentrations that can be reached in vivo in the pulmonary tree.

At the same time, the biofilms thus treated were submitted to sonication in order to disperse the cells that were still adhering, and these were seeded on rich medium after suitable dilution for calculating the vital elements surviving the various treatments.

### EXAMPLE 2

### Effect of NAC on the Formation and Disruption of Biofilms and on Bacterial Viability

In the case of S. aureus, NAC did not cause visible changes in bacterial growth, even at the maximum concentration used (8 mg/ml). However, NAC inhibited glycocalyx production in all the strains. At a concentration of 8 mg/ml, reductions of the biofilm greater than 50% (68.2 and 56.3%) were observed in 2 strains (S. aureus 1393 and 1890). In the other two strains, S. aureus 1876 and 1880, biofilm production decreased by 30.3% and 41.1%, respectively.

At the maximum concentration used, NAC disrupts the biofilm that formed after 5 hours of bacterial growth (initial maturation) by more than 50% (61.1%, 57.3%, 58.6%) in 3 strains (1393, 1880 and 1890). For S. aureus 1876 the calculated reduction was 32.5%. NAC reduced the quantity of mucopolysaccharide material of the biofilms fully consolidated at maturation by more than 50% in all four strains assayed (62%, 60.5%, 58.1% and 65.5%).

At the maximum concentration used, both on the cells of biofilm at initial maturation and on those of the biofilm at consolidated maturation, NAC caused reductions in colony forming units per ml (CFU/ml) varying from 33.7% to 91% for the cells belonging to the youngest biofilms and from 33.7% to 70.6% for the mature biofilms.

### EXAMPLE 3

### Effect of NAC Combined with Thiamphenicol on Biofilm Disruption and on Bacterial Viability

On biofilms in the initial stage, the effect of NAC combined with TAPh was greater, the higher the concentrations of the two molecules.

At the maximum concentrations used (32 mg/l of TAPh) the combination caused disruption of the initial biofilms varying from 63% (S. aureus 1876) to 78.5% (S. aureus 1890).

The percentage disruption of the fully consolidated biofilms varied from 59.7% (S. aureus 1876) to 67.5% (S. aureus 1393). These reduction values are greater than those obtained using NAC (32.5-65.5% and 58.1-62%) on its own.

Very similar results were obtained on combining NAC with TAPh at 16 and 8 mg/l.

On the vital elements of the biofilm, the effect of the two molecules combined, at the maximum concentrations used, caused a decrease of the CFU/ml values of 1 or 2 logarithms in all the strains assayed.

1. A process for preparing a pharmaceutical composition containing N-acetyl-cysteine for the preventive or disruptive treatment of biofilms produced by Staphylococcus aureus and by other pathogen agents of the respiratory system.

2. A process for preparing a pharmaceutical composition containing thiamphenicol useful for increasing the activity of N-acetyl-cysteine in inhibiting or disrupting the biofilm produced by Staphylococcus aureus and by other pathogen agents of the respiratory system.

3. A pharmaceutical composition containing N-acetyl-cysteine and thiamphenicol and use thereof in inhibiting or disrupting biofilms produced by Staphylococcus aureus and by other pathogen agents of the respiratory system.

4. A process for preparing a pharmaceutical composition containing thiamphenicol glycinate acetylcysteinate useful for inhibiting or disrupting biofilms produced by Staphylococcus aureus and by other pathogen agents of the respiratory system.
